(19) United States
(12) Patent Application Publication
(10) Pub. No.: US 2006/0233857 A1
(43) Pub. Date: Oct. 19, 2006

(54) Degradable elastomeric network

(76) Inventors: Brian G. Amsden, Kingston (CA); Gauri Prasad Misra, Hershey, PA (US)

Correspondence Address: Stephen J. Scribner;PARTEQ Innovations, Room 1625, Biosciences Complex, Queen's University at Kingston, Kingston, ON, K7L 3N6, CA

(21) Appl. No.: 11/403,203

(22) Filed: Apr. 13, 2006

Related U.S. Application Data
(60) Provisional application No. 60/671,093, filed on Apr. 14, 2005.

(30) Foreign Application Priority Data
Apr. 14, 2005 (CA) 2,504,076

(51) Int. Cl. A61K 48/00; A61K 39/395; A61K 39/00; A61K 39/12; A61F 2/00

(52) U.S. Cl. 424/426; 424/204.1; 424/133.1; 514/2; 514/44

(57) ABSTRACT

One aspect of the invention provides a degradable/biocompatible elastomer. The elastomer comprises a degradable cross-linked network of a hydrophobic, hydrolysable amorphous star polymer and a hydrophilic, biocompatible polymer. The network may be crosslinked thermally or by irradiation. In a preferred embodiment, the elastomer is used for a drug delivery system, and is particularly useful for delivery of peptide and protein drugs.

### RELATED APPLICATIONS

This application claims the benefit of the filing date of U.S. Patent Application Ser. No. 60/671,093, filed on Apr. 14, 2005, the contents of which are incorporated herein by reference in their entirety.

### FIELD OF THE INVENTION

This invention relates to biodegradable/biocompatible elastomeric materials. Such materials are suitable for use as implantable medical devices. In particular, this invention relates to cross-linked biodegradable/biocompatible elastomeric materials suitable for use as implantable drug delivery devices.

### BACKGROUND OF THE INVENTION

Biodegradable and/or biocompatible polymeric materials are widely used in the manufacture of implantable medical devices, including drug delivery depots. Elastomeric polymers are advantageously used in such applications because they are less likely to produce tissue irritation at the implant site and, for setting elastomers, they maintain their geometric dimensions during release and degradation. Cured elastomers can be prepared using heat or photo-irradiation to form covalent linkages between polymer chains (see, for example, U.S. Pat. No. 6,984,393, issued Jan. 10, 2006). However, for drug delivery devices involving the entrapment of temperature-sensitive drugs such as peptides or proteins, a thermo-setting elastomer is unsuitable.

Many peptide and protein drugs, e.g. cytokines, are effective at very low concentrations, have very short biological half-lives, act in a paracrine fashion, require long-term delivery and are readily degraded when administered by conventional routes. For these reasons considerable effort has been devoted to the development of formulations for prolonged localized delivery, most of which have focused on the use of biodegradable polymers as delivery vehicles (Amkraut et al., Adv. Drug Delivery Rev. 1990, 4:255-276; Gombotz et al., Bioconjugate Chem. (1995) 6:332-351; Sinha et al., J. Control. Rel. (2003) 90:261-280; Schwendeman et al., Peptide, protein, and vaccine delivery from implantable polymeric systems. In: Controlled Drug Delivery: Challenges and Strategies, ed.: Park, K., ACS: Washington, D.C., (1997)). In particular, the development of biodegradable microparticle formulations has received much attention.

Typically, in such delivery systems the drug is incorporated as a solid particle dispersed throughout the polymer matrix. The drug is released by dissolution and diffusion of surface resident particles and any particles in contact with those at the surface. Subsequent release for biodegradable systems then proceeds through the creation of micropores within the device as the polymer begins to hydrolyze. For low drug loadings, only a fraction of the drug can be released by diffusion, and so the majority of the drug is released through the creation of pores by polymer degradation. This generally results in a biphasic release pattern, with release by diffusion occurring first and reaching a plateau, and erosion-controlled release occurring after a lag period. Thus, for drugs that should be released at low concentrations but within a reasonable time frame, use of a hydrophobic polymer matrix is a poor choice, as drug release rates are controlled by the interconnectedness of the particles within the matrix (Gombotz et al., Bioconjugate Chem. (1995) 6:332-351).

One way to increase the amount of drug released in the diffusional phase is by including physiologically innocuous, water soluble excipients in the delivery device. Such excipients increase the porosity of the device by dissolving to generate pores and may also enhance polymer degradation by increasing water absorption into the device. The incorporated drug is released by diffusion through the pores. The inclusion of water soluble excipients may also eliminate the biphasic release pattern. However, a combination of enhanced total fraction released and a sustained constant release rate is not possible with this approach, because the release rate increases as the porosity of the device increases.

Other approaches have included the use of block thermoplastic copolymers, containing a water-soluble polymer block (e.g., poly(ethylene glycol)) and a hydrophobic polymer block, typically poly(D,L-lactide). Using these block copolymers, the protein is loaded into the polymer device by dissolving the polymer in a suitable organic solvent and then using processes such as emulsification, and solvent casting (Kissel et al., J. Control. Rel. (1996) 39:315-326; Bezemer et al., J. Control. Rel. (2000) 64:179-192). This approach has been demonstrated to be capable of generating constant protein release rates. However, this approach often results in a significant initial burst release of drug, and/or denaturation of the drug during device fabrication.

Polymeric materials having a temperature-dependent drug release profile were disclosed by Aoyagi et al. (J. Control. Rel. (1994) 32:87-96), and Nagase et al. (U.S. Pat. No. 5,417,983). Temperature dependence of the drug release was obtained from star polymers having specific crystallinity.

### SUMMARY OF THE INVENTION

In a first aspect, the invention provides a degradable delivery system for delivering an agent, comprising: a biocompatible degradable cross-linked network of: a hydrophobic, hydrolysable amorphous star polymer; and a hydrophilic polymer; and an agent distributed within the network.

The star polymer may comprise at least one monomer, said at least one monomer capable of forming a degradable linkage to another monomer. The at least one monomer may be selected from the group consisting of lactones, carbonates, and cyclic amides, and combinations thereof. The at least one monomer may be selected from valerolactone, caprolactone, dioxepanone, lactide, glycolide, trimethylene carbonate, and O-benzyl-L-serine.

In certain embodiments, the polymers may further comprise one or more cross-linkable groups on the polymer chain termini.

The cross-linking may be initiated thermally or by irradiation. The delivery system may further comprise a photo-cross-linkable group selected from acrylate, coumarin, thymine, cinnamates, diacrylate, oligoacrylate, methacrylate, dimethacrylate, and oligomethacrylate.

The cross-linked network may be formed through action of an initiator.

In certain embodiments of the delivery system, the polymer chain termini may contain a carbon-carbon double bond capable of cross-linking and polymerizing polymers.

In certain embodiments, the initiator may be a free radical initiator selected from acetophenone derivatives, camphorquinone, Irgacure® (1-hydroxy-cyclohexyl-phenyl-ketone, 1-[4-(2-hydroxyethoxy)-phenyl]-2-hydroxy-2-methyl-1-propane-1-one, 2,2-dimethoxy-1,2-diphenylethan-1-one, or 2-methyl-1-[4-(methylthio)phenyl]-2-(4-morpho-linyl)-1-propanone, 2,2-dimethyl-2-phenylacetaphenone, 2-methoxy-2-phenylacetaphenone), Darocur® (1-[4-(2-hydroxyethoxy)-phenyl]-2-hydroxy-2-methyl-1-propane-1-one or 2,4,6-trimethylbenzoyl-diphenyl-phosphineoxide), eosin dye, potassium persulfate, with or without tetraamethyl ethylenediamine; benzoylperoxide, with or without triethanolamine; and ammonium persulfate with sodium bisulfite.

In some embodiments of the delivery system, the star polymer has a glass transition temperature (T_{g}) below room temperature. The star polymer may comprise star-poly(ε-caprolactone-co-D,L-lactide).

In some embodiments of the delivery system, the hydrophilic polymer may be selected from poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates such as hyalyuronic acid, chitosan, dextran, heparan sulfate, heparin, alginate, and proteins such as gelatin, collagen, albumin, ovalbumin, and polyamino acids.

In some embodiments of the delivery system, the hydrophilic polymer may comprise poly(ethylene glycol)diacrylate.

The hydrophobic polymer may form greater than 70% by weight of the total polymer mass, and the rate of agent release increases as the content of hydrophobic polymer decreases.

In some embodiments, the agent may be a drug, a peptide, or a protein. In other embodiments, delivery system may be a medical device, may be adapted for implant in a subject, and may be biodegradable.

In a second aspect, there is provided a method of preparing a biocompatible degradable delivery system for delivering an agent, comprising: combining a hydrophobic, hydrolysable amorphous star polymer and a hydrophilic polymer to create a mixture; adding an agent to the mixture; and subjecting the mixture to photo-irradiation to create a degradable cross-linked solid network.

In some embodiments, the mixture may be disposed in a mold prior to photo-irradiation. In some embodiments, the star co-polymer may comprise at least one monomer, said at least one monomer capable of forming a biodegradable linkage to another monomer.

In some embodiments, the monomer may be capable of undergoing polymerization through a ring-opening reaction or a condensation reaction.

In some embodiments, the at least one monomer may be selected from the group consisting of lactones, carbonates, and cyclic amides, including valerolactone, caprolactone, dioxepanone, lactide, glycolide, trimethylene carbonate, and O-benzyl-L-serine.

In some embodiments, the star polymer may further comprise one or more photo-cross-linkable groups on the polymer chain termini, wherein the photo-cross-linkable group may be selected from acrylate, coumarin, thymine, cinnamate, diacrylate, oligoacrylate, methacrylate, dimethacrylate, and oligomethacrylate.

In some embodiments, the cross-linked network may be formed through action of an initiator.

In some embodiments, the termini of the polymers may contain a carbon-carbon double bond capable of cross-linking and polymerizing polymers.

In some embodiments, the initiator may absorb photons to form a free radical which reacts with an allyl group of the photo-cross-linkable group. The initiator may be selected from acetophenone derivatives, camphorquinone, Irgacure® (1-hydroxy-cyclohexyl-phenyl-ketone, 1-[4-(2-hydroxyethoxy)-phenyl]-2-hydroxy-2-methyl-1-propane-1-one, 2,2-dimethoxy-1,2-diphenylethan-1-one, or 2-methyl-1-[4-(methylthio)phenyl]-2-(4-morpho-linyl)-1-propanone, 2,2-dimethyl-2-phenylacetaphenone, 2-methoxy-2-phenylacetaphenone), Darocur® (1-[4-(2-hydroxyethoxy)-phenyl]-2-hydroxy-2-methyl-1-propane-1-one or 2,4,6-trimethylbenzoyl-diphenyl-phosphineoxide), and eosin dye.

In a preferred embodiment, the star polymer may comprise star-poly(ε-caprolactone-co-D,L-lactide).

In some embodiments, the star polymer may be end-functionalized with a vinyl monomer.

In some embodiments, the hydrophilic polymer may be selected from poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates such as hyalyuronic acid, chitosan, dextran, heparan sulfate, heparin, alginate, and proteins such as gelatin, collagen, albumin, ovalbumin, and polyamino acids.

In a preferred embodiment, the hydrophilic polymer may comprise poly(ethylene glycol)diacrylate.

In some embodiments, the hydrophobic polymer may form greater than 70% by weight of the total polymer mass.

In some embodiments, the agent may be a drug, a peptide, or a protein.

In a third aspect of the invention there is provided a method of delivering a drug to a subject, comprising: providing the drug in a delivery system comprising a biocompatible degradable cross-linked network of a hydrophobic, hydrolysable amorphous star polymer and a hydrophilic polymer; and disposing the delivery system in the subject.

In some embodiments, wherein the drug may be a peptide or a protein.

In a fourth aspect of the invention there is provided a biocompatible degradable elastomer, comprising: a degradable cross-linked network of: a hydrophobic, hydrolysable amorphous star polymer; and a hydrophilic polymer.

In some embodiments, the elastomer may be biodegradable.

In some embodiments, the cross-linking may be photo-cross-linking.

In some embodiments, pores of the elastomer may be connected.

According to another aspect of the invention there is provided a degradable elastomer, comprising: a biocompatible degradable cross-linked network of:

(i) a hydrophobic, hydrolysable amorphous star polymer; and (ii) a hydrophilic polymer;

wherein one of the hydrophobic polymer or the hydrophilic polymer includes two or more cross-linkable groups on the polymer chain terminus, and the other of the hydrophobic polymer or the hydrophilic polymer includes one or more cross-linkable groups on the polymer chain terminus.

The star polymer may comprise at least one monomer, the at least one monomer capable of forming a degradable linkage to another monomer. The at least one monomer may be selected from lactones, carbonates, and cyclic amides, and combinations thereof. The at least one monomer may selected from valerolactone, caprolactone, dioxepanone, lactide, glycolide, trimethylene carbonate, and O-benzyl-L-serine. In a preferred embodiment the star polymer comprises star-poly(ε-caprolactone-co-D,L-lactide).

The hydrophilic polymer may be selected from poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates such as hyalyuronic acid, chitosan, dextran, heparan sulfate, heparin, alginate, and proteins such as gelatin, collagen, albumin, ovalbumin, and polyamino acids. In a preferred embodiment the hydrophilic polymer comprises poly(ethylene glycol)diacrylate.

According to another aspect of the invention there is provided a method of preparing a biocompatible degradable elastomer, comprising:

providing a hydrophobic, hydrolysable amorphous star polymer and a hydrophilic polymer, one of the hydrophobic polymer or the hydrophilic polymer including two or more cross-linkable groups on the polymer chain terminus, and the other of the hydrophobic polymer or the hydrophilic polymer including one or more cross-linkable groups on the polymer chain terminus;

combining the hydrophobic, hydrolysable amorphous star polymer and the hydrophilic, biocompatible polymer; and

cross-linking the hydrophobic, hydrolysable amorphous star polymer and the hydrophilic, biocompatible polymer to create a degradable cross-linked elastomer.

The method may further comprise combining the hydrophobic, hydrolysable amorphous star polymer and the hydrophilic polymer in a mold prior to cross-linking.

The star polymer may comprise at least one monomer, said at least one monomer capable of forming a biodegradable linkage to another monomer. The monomer may be capable of undergoing polymerization through a ring-opening reaction or a condensation reaction. The at least one monomer may selected from lactones, carbonates, and cyclic amides. The at least one monomer may selected from valerolactone, caprolactone, dioxepanone, lactide, glycolide, trimethylene carbonate, and O-benzyl-L-serine.

In one embodiment, the method may further comprise forming the cross-linked network through action of an initiator, wherein the initiator absorbs energy to form a free radical which reacts with an allyl group of the cross-linkable group.

The cross-linkable group may comprise a photo-cross-linkable group selected from acrylate, coumarin, thymine, cinnamate, diacrylate, oligoacrylate, methacrylate, dimethacrylate, and oligomethacrylate.

The initiator may be a photo-initiator selected from acetophenone derivatives, camphorquinone, Irgacure® (1-hydroxy-cyclohexyl-phenyl-ketone, 1-[4-(2-hydroxyethoxy)-phenyl]-2-hydroxy-2-methyl-1-propane-1-one, 2,2-dimethoxy-1,2-diphenylethan-1-one, or 2-methyl-1-[4-(methylthio)phenyl]-2-(4-morpho-linyl)-1-propanone, 2,2-dimethyl-2-phenylacetaphenone, 2-methoxy-2-phenylacetaphenone), Darocur® (1-[4-(2-hydroxyethoxy)-phenyl]-2-hydroxy-2-methyl-1-propane-1-one or 2,4,6-trimethylbenzoyl-diphenyl-phosphineoxide), and eosin dye.

In another embodiment, the initiator may be a thermal initiator selected from potassium persulfate, with or without tetraamethyl ethylenediamine; benzoylperoxide, with or without triethanolamine; and ammonium persulfate with sodium bisulfite.

In a preferred embodiment, the star polymer comprises star-poly(ε-caprolactone-co D,L-lactide).

The hydrophilic polymer may be selected from poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates such as hyalyuronic acid, chitosan, dextran, heparan sulfate, heparin, alginate, and proteins such as gelatin, collagen, albumin, ovalbumin, and polyamino acids.

In a preferred embodiment, the hydrophilic polymer comprises poly(ethylene glycol) diacrylate.

According to another aspect of the invention there is provided an implantable delivery system for delivering a pharmaceutical agent to a subject, comprising the degradable elastomer as described above and the agent distributed within the network, wherein the network provides controlled release of the agent. The agent may be a therapeutic compound, pharmaceutical, biopharmaceutical, medicament, hormone, peptide, protein, nucleic acid, vector, virus, antigen, or antibody, or combination thereof. In one embodiment, rate of release of the agent increases as the content of hydrophobic polymer in the network decreases.

According to another aspect of the invention there is provided a device comprising the degradable elastomer as described above. The device may be a biomedical device selected from a needle, stent, catheter, and a scaffold.

According to another aspect of the invention there is provided a method of delivering a pharmaceutical agent to a subject, comprising: providing the agent in the implantable delivery system described above; and implanting the delivery system in the subject. The agent may be a therapeutic compound, pharmaceutical, biopharmaceutical, medicament, hormone, peptide, protein, nucleic acid, vector, virus, antigen, or antibody, or combination thereof.

### BRIEF DESCRIPTION OF THE DRAWINGS

Preferred embodiments of the invention will now be described, by way of example, with reference to the drawings, wherein:

FIG. 1 is a plot showing the influence of weight percent of poly(ethylene glycol) diacrylate (PEGD) incorporated into networks on vitamin B12 release from cylinders prepared using acrylated star co-polymer (ASCP) 1000 (see Example 1 for details). The cylinders had a diameter of 3.5 mm and the vitamin B12 particle size was <100 μm. The solid lines represent linear regressions to the data over the region indicated.

FIG. 2 is a plot showing the influence of cylinder diameter on vitamin B12 release. The cylinders were prepared using ASCP 1000 and contained 10% PEGD. The vitamin B12 particle size was <100 μm. The solid lines represent linear regressions to the data over the region indicated.

FIG. 3 is a plot showing the effect of ASCP molecular weight on vitamin B12 release. The PEGD content was 10%, the cylinder diameter was 1.8 mm, and the vitamin B12 particle size was <100 μm.

FIG. 4 is a plot showing the effect of vitamin B12 particle size on release from 1.8 mm cylinders prepared using ASCP 2700 containing 10% PEGD.

FIG. 5 is a plot showing the volume change of vitamin B12 loaded cylinders with release time. The data is expressed as the volume at time t, Vₜ, divided by the initial volume, Vₒ. (A) Cylinders prepared using ASCP 1000 containing 10 w/w % PEGD. (B) Cylinders prepared using 10 w/w % PEGD and varying ASCP molecular weight. The initial cylinder diameter was 1.8 mm and the vitamin B12 particle size was <100 μm.

FIG. 6 is a plot showing in vitro mass loss with time for cylindrical networks prepared with ASCP 1000 and varying amounts of PEGD. The data is expressed as mass at time t, mₜ, divided by the initial mass, mₒ.

FIG. 7 is a plot showing influence of PEGD molecular weight on vitamin B12 release from elastomer cylinders.

FIG. 8 is a plot showing release of goserelin acetate and vitamin B12 from cylindrical matrices prepared from 90% ASCP 2700 and 10% PEGD 24000. The compound loading in each case was 1 w/w %.

### DETAILED DESCRIPTION OF PREFERRED EMBODIMENTS

In accordance with a broad aspect of the invention, there is provided a degradable, preferably biodegradable, and/or biocompatible cross-linked elastomeric polymer. The elastomeric polymer, also referred to herein as “elastomer”, is useful in applications such as, for example, biomaterials and biomedical devices, where it can be used in treatment of human and non-human subjects, and in plants, and in applications such as human, non-human, and plant tissue engineering and tissue culture. Elastomers of the invention can be formed into films, rods, screws, needles, stents, catheters, or other structures with or without incorporated fibres; implantable drug/agent delivery systems, in which an agent is disposed in the elastomer and released therefrom; film coatings for pills; coatings on biomedical devices such as needles, stents, and catheters; as well as other applications such as rubber tougheners for ceramic devices. The elastomer may be formed into devices such as scaffolds for tissue engineering and tissue restoration of soft tissue, connective tissue, and bone, in vitro and in vivo. Also, the elastomer may be provided as a coating on devices such as scaffolds for tissue engineering and tissue restoration of soft tissue, connective tissue, and bone, in vitro and in vivo.

As used herein, the terms “drug” and “agent” are used interchangeably and are intended to refer to any therapeutic compound, pharmaceutical, or biopharmaceutical, which may include, for example, a medicament, hormone, peptide or protein, nucleic acid, vector, virus, antigen, or antibody, or any combination of these, incorporated or entrapped in an elastomer of the invention and released therefrom. Examples of applications of the elastomer include, but are not limited to, medicine, veterinary science, immunology, transgenics, management of allergies, and birth control, as well as other applications where chronic or long-term delivery of an agent is required.

Other applications of the elastomer where delivery of an agent encapsulated in, or loaded into, a biodegradable/biocompatible polymer is required, or would be beneficial, include, for example, agriculture. An elastomer of the invention may be loaded with one or more agents such as a fertilizer or pesticide. Application of the loaded elastomer to a crop results in sustained delivery of the one or more agents. Such delivery helps to avoid over-fertilizing of crops, and reduces or eliminates the need for repeated applications of such agents.

Depending on the properties of the agent loaded into the elastomer, and the desired delivery rate of the agent, an excipient, as described below, can be used together with such agent. Also depending on the properties of the agent loaded into the elastomer, it may be desirable to protect the agent by treating the agent during or prior to loading into the elastomer. For example, when the agent is a drug, the drug may be co-lyophilized with a protecting agent prior to loading.

As used herein, the term “degradable” is intended to refer to a substance that can be chemically degraded or decomposed by natural effectors, for example, via weather or biological processes (i.e., biodegradable) such as physiological temperature, pH, and/or enzyme activity. For example, degradation may occur by hydrolysis, which can occur chemically and/or in a biological system. Biological processes can take place within an organism or outside of an organism.

As used herein, the term “biocompatible” is intended to refer to a substance having substantially no known toxicity to or adverse affects on biological processes. The substance can be a compound in its original state or one or more components or products of the compound as the compound biodegrades.

In one embodiment of the invention, there is provided a biodegradable/biocompatible elastomeric polymer. The elastomer comprises a degradable cross-linked network of a hydrophobic, hydrolysable amorphous star co-polymer and a hydrophilic polymer. In embodiments where the elastomer is used to deliver an agent, such as a drug, the drug is distributed either as drug particles throughout or is dissolved within the elastomer. The star polymer and the hydrophilic polymer are modified such that they contain one or more cross-linkable groups on the polymer chain termini.

It should be noted that cross-linking may be accomplished using thermal or irradiation polymerization initiator systems. Thermal initiator systems that are unstable at temperatures less than about 60° C., preferably around 37° C., and that initiate free radical polymerization at physiological temperatures include, for example, potassium persulfate, with or without tetraamethyl ethylenediamine; benzoylperoxide, with or without triethanolamine; and ammonium persulfate with sodium bisulfite. However, an irradiation system, particularly involving photo-cross-linking, is the preferred method of cross-linking, because it can be accomplished very rapidly, with minimal heat generation (Sawhney et al., Macromolecules (1993) 26:581-587), and therefore may not lead to degradation of an agent, such as a peptide drug, to be entrapped.

Photo-cross-linking is also preferable because it allows for formation of elastomeric biomedical devices and agent delivery systems in vivo. For example, the polymer mixture may be injected into a subject, and then polymerized by photo-cross-linking to obtain the elastomer in situ. Depending on the particular situation, the photo-cross-linking light maybe applied through the skin, via a fibre optic cable, or otherwise as appropriate. Alternatively, the polymer mixture may be implanted into a subject during surgery, and then polymerized by photo-cross-linking to obtain the elastomer prior to closing the incision. Such a system allows an elastomeric device to be custom fitted to a particular location or physiological situation, and allows the physician to verify the correct placement of the implant prior to closing the incision.

Suitable star polymers may be prepared from any monomer capable of forming a biodegradable linkage to another monomer and capable of undergoing polymerization through a condensation reaction, or preferably through a ring-opening reaction. Preferably, the monomer or monomers are chosen so as to form an amorphous star polymer. Such monomers include, for example, lactones, carbonates, cyclic amides (e.g., polyester amides, polyamides), and combinations thereof. Examples of such monomers are valerolactone, caprolactone, dioxepanone, lactide, glycolide, trimethylene carbonate, and O-benzyl-L-serine.

A suitable cross-linkable group may be any group with an accessible carbon-carbon double bond that can undergo free radical polymerization. Examples of cross-linkable groups are coumarin, thymine, cinnamates, acrylates, including, for example diacrylates, oligoacrylates, methacrylates, dimethacrylates, and oligomethacrylates. Cross-linkable groups may be substituted or unsubstituted. Preferred cross-linkable groups are acrylates which cross-link faster than methacrylates. The photo-cross-linking reaction may be initiated by a compound which absorbs photons to form a free radical which reacts with the allyl group of the photo-cross-linkable group. Examples of such an initiator are acetophenone derivatives (2,2-dimethyl-2-phenylacetaphenone, 2-methoxy-2-phenylacetaphenone), camphorquinone, Irgacure® (1-hydroxy-cyclohexyl-phenyl-ketone, 1-[4-(2-hydroxyethoxy)-phenyl]-2-hydroxy-2-methyl-1-propane-1-one, 2,2-dimethoxy-1,2-diphenylethan-1-one, or 2-methyl-1-[4-(methylthio)phenyl]-2-(4-morpho-linyl)-1-propanone), Darocur® (1-[4-(2-hydroxyethoxy)-phenyl]-2-hydroxy-2-methyl-1-propane-lone or 2,4,6-trimethylbenzoyl-diphenyl-phosphineoxide), and eosin dye. The wavelength (e.g., visible, ultraviolet (UV)) and intensity of light used for the photo-cross-linking reaction depend on the specific initiator used.

The hydrophilic polymer may be crystalline, non-crystalline, or semi-crystalline and may be selected from poly(ethylene glycol) (PEG), poly(ethylene oxide), poly(vinyl alcohol), poly(vinylpyrrolidone), poly(ethyloxazoline), poly(ethylene oxide)-co-poly(propylene oxide) block copolymers, polysaccharides or carbohydrates such as hyaluronic acid, chitosan, dextran, heparan sulfate, heparin, alginate, proteins such as gelatin, collagen, albumin, ovalbumin, or polyamino acids. The matrix may also be made from a diacrylated triblock polymer such as poly(D,L-lactide)-block-poly(ethylene glycol)-block-poly(D,L-lactide). The use of such a triblock polymer instead of a homopolymer of PEG is expected to increase the degradation rate of the elastomer.

In a preferred embodiment, the hydrophobic star polymer is star-poly(ε-caprolactone-co-D,L-lactide) that has been end-functionalized with a vinyl monomer, and the hydrophilic polymer is comprised of poly(ethylene glycol) that has been end-functionalized with a vinyl monomer.

The elastomer, or delivery device comprising the elastomer, may be prepared by first dissolving the star co-polymer and the hydrophilic polymer in a suitable solvent. In embodiments where the elastomer is for a delivering an agent such as a drug, the drug, in particulate form, is then added to the solution to create a suspension. In some embodiments, the solvent may not be necessary. A photo-initiator is then added, and mixed throughout the suspension. To obtain an elastomer device of desired shape (e.g., cylindrical), the suspension may be poured into a suitable mold and immediately subjected to photo-irradiation. A cross-linked, solid network is formed that entraps the solid drug particles. Residual solvent is removed by evaporation.

The rate of drug release is controlled by the weight ratio of hydrophobic polymer present. As the hydrophobic polymer content decreases, the release rate increases. A long-term continuous release of the entrapped agent, such as a peptide or protein drug, is achieved when the hydrophobic polymer forms greater than 70% by weight of the total polymer mass. The molecular weight of the hydrophilic polymer may also influence the release rate of an entrapped drug or agent. For example, when diacrylated poly(ethylene glycol) (PEGD) is used, the release rate increases with increasing molecular weight of the PEGD.

Requirements for the formation of a useful elastomer using a star polymer as a prepolymer are that the prepolymer has a glass transition temperature (T_{g}) below physiological temperature (e.g., 37° C.), and preferably below room temperature, and is amorphous. Glass transition temperature is the temperature at which a polymer undergoes a phase transition from a glassy state to a rubbery state upon heating. It is the temperature where the molecules of a polymeric solid begin to move relative to one another, yielding a substance that behaves like a rubber, rather than a brittle glass.

Thus, star polymers in which at least one monomer has a very low glass transition temperature are the most suitable. An example of a monomer suitable for use in accordance with the invention is ε-caprolactone (T_{g}=−60° C.). Such monomer can be used to prepare a star polymer, such as a star co-polymer, with another monomer such as D,L-lactide, even though the glass transition temperature of D,L-lactide is 68° C.

In preparing a star polymer from one or more species of monomers, an initiator is used. The initiator can be any polyol, such as, for example, glycerol, pentaerythritol, and xylitol.

As noted above, a star polymer in accordance with the invention can comprise one or more species monomer. In general, the properties (e.g., physical properties such as strength, Young's modulus, etc., and degradation kinetics) of the elastomer are determined to a large extent by the composition of the star polymer, and, where two or more monomers are employed, by the molar ratios of the monomers. For example, where an elastomer having more rapid biodegradation kinetics is desired, a monomer that either biodegrades more rapidly, and/or is more hydrophilic, should be chosen for incorporation into the star polymer. Thus, in the case of a polymer of ε-caprolactone and D,L-lactide, the relative proportions of ε-caprolactone and D,L-lactide should be controlled so as to produce a polymer that is amorphous. However, increasing the D,L-lactide content increases the biodegradation rate of the elastomer. It will be appreciated that, in accordance with the invention, an elastomer having a desired set of physical properties, including biodegradation rate, can be prepared by designing a star polymer with a specific architecture, and controlling the amount of cross-linking agent used. Moreover, such an elastomer is easily reproduced.

In embodiments where elastomers of the invention are loaded with a pharmaceutical agent and used for implantable delivery devices for the agent, the delivery rate of the agent will be a function of the hydrophobic star polymer content. For example, we have found that when the content of the hydrophobic star polymer is less than about 70 w/w % of the elastomer, and the elastomer is loaded with an agent having a molecular weight of about 1355 g/mol (i.e., the average weight of many peptide drugs), substantially all of the agent is delivered from the elastomer in about four days (see the below example). However, when the content of the hydrophobic star polymer is about 90 w/w % of the elastomer, and the elastomer is loaded with the same agent, delivery of the agent is slower and more linear over the delivery period. Thus, it is preferred that the content of the hydrophobic star polymer is at least about 70% by weight of the total polymer mass, more preferably about 70% to about 90%. Of course, the delivery rate will also depend on the molecular weight of the agent, with larger agents having slower delivery.

It is noted that Aoyagi et al. (J. Control. Rel. 32:87-96, 1994), see also U.S. Pat. No. 5,417,983, relates to a photo cross-linked polymeric material for use as a drug delivery vehicle, whose permeability to a drug changes with temperature. The temperature responsiveness was obtained by preparing the material from star polymers specifically designed to possess crystallinity. The crystallinity provided the temperature response through melting of the crystalline regions at elevated temperatures. Crystallinity of the polymers was ensured by using monomers known to produce crystalline homopolymers, i.e., ε-caprolactone and L-lactide. Semi-crystalline star polymers were formed using three strategies: 1) by the homopolymerization of α-caprolactone, 2) by the preparation of star-block polymers using these monomers, and 3) by using a low ratio of co-monomer (less than 15 mol %) when preparing a random star-copolymer using these monomers. Such polymers are distinct from elastomers of the invention, in that elastomers of the invention are prepared from amorphous (i.e., non-crystalline) star polymers. Further, when an elastomer of the invention is used as a vehicle to deliver an agent, release of the agent from the elastomer is independent of thermal transitions of the elastomer.

Advantages of the photo-cross-linked elastomer include:

1. The biodegradable elastomer can be prepared at room or physiologic temperature and thus may be prepared in vivo.

2. The low temperature during preparation avoids thermal denaturation of peptide and protein drugs.

3. The prepolymer is a star polymer which has a reduced viscosity, which allows for easier insertion into molds for part manufacture, and thus may be processed at lower temperatures than linear counterparts.

4. The prepolymer is amorphous (non-crystalline) and produces an amorphous elastomer which degrades at a more homogeneous rate than would a thermoplastic elastomer which relies on crystalline blocks of homopolymer sections of the backbone to provide cross-links (amorphous regions degrade first, then the crystalline regions which degrade more slowly).

5. Because of its homogeneous degradation rate, the elastomer maintains its physical properties for a longer time period (provides a linear decrease in strength with respect to mass loss during degradation).

Further, in embodiments where the elastomer is provided as a drug delivery device, the elastomer advantageously provides the combination of low drug loading, minimal burst effect, nearly constant release, and 100% drug entrapment. None of the known prior drug delivery systems provides this combination of advantages.

In particular, the delivery system of the invention provides for a very low (e.g., 1 w/w %) amount of drug loaded into the device, with virtually complete release, as well as near zero order for a prolonged period of time. Many drugs, such as growth factors and cytokines, have very low minimum effective concentrations and are required to be delivered locally for a prolonged period of time. Even with an osmotic delivery system, the solids (drugs or drug+filler) loading is at least 10% to accomplish the same objective. Also, with the invention, release of drug is not polymer degradation dependent, which may be advantageous for peptide and protein delivery because there would likely be no drug degradation due to liberation of polymer degradation products. In contrast, the most commonly used degradable polymer, PLGA, relies on a degradation mechanism, along with device porosity, to achieve the same type of release profile (van de Weert et al., Pharm. Res. (2000), 17:1159-1167). However, this system has been shown to result in degradation of multiple proteins.

As noted above, the biodegradable/biocompatible cross-linked elastomer of the invention is particularly well suited for drug delivery devices, such as controlled release devices. Advantages of such an elastomeric device surgically implanted in a subject include: administration of a drug at a desired location, with sustained slow release with minimal burst effect and depot effect, so that the total dosage administered to a subject can be reduced, and the potential for systemic side effects is reduced; further surgery to retrieve the delivery device is avoided because the device is biodegradable and biocompatible; and the elastomer may protect the drug from degradation until it is released.

Lipophilic drugs, (for example, but not limited to bupivacaine, benzocaine, lidocaine, camptothecin, paclitaxel, etoposide, vincristine, vinblastine, vitamin D, tacrolimus, hydrocortisone, nitroglycerin, fentanyl, estradiol, testosterone, cortisone and other corticosteroids), hydrophilic drugs (for example, but not limited to pilocarpine nitrate, aspirin, ibuprofen, potassium chloride, ascorbic acid), and peptide and protein drugs (for example, but not limited to cytokines such as interferons, interleukins, granulocyte macrophage colony stimulating factor, insulin, erythropoeitin, human growth hormone, epidermal growth factor, vascular endothelial growth factor, basic fibroblast growth factor), and combinations thereof, may be loaded into a delivery device using an elastomer of the invention.

In some embodiments an excipient is included in addition to a drug or drugs. Excipients, which may be bulking agents or osmotagens, are physiologically inert, and may enhance delivery or increase the rate of delivery of a drug by generating osmotic pressure within the elastomer. The mechanism of osmotically controlled release is as follows: Upon immersion into an aqueous medium, drug release begins as water vapor penetrates the polymer matrix until it reaches a polymer encapsulated particle, hereafter referred to as a capsule. The water phase-separates and dissolves the solid drug at the polymer/drug interface, forming a saturated solution of drug and excipient particles. Under the reduced water activity gradient, water is drawn into the capsule, causing it to swell. If the osmotic pressure is great enough, the polymer capsule wall ruptures. Due to the relaxation process of the elastomer, the capsule wall slowly collapses and the solution of drug and excipient particles is forced out through the rupture. This rupture and collapse process results in the drug being released at an almost constant rate. Osmotic drug delivery from monolithic polymer devices has been described (Michaels et al., U.S. Pat. No. 4,117,256; Di Colo, Biomaterials. 13(12):850-856, 1992; Amsden et al., J. Control. Rel. 30:45-56, 1994) using non-biodegradable polymers such as poly(ethylene-vinylacetate) and silicone.

The delivery of peptide and protein drugs may be problematic due to the sensitivity of such drugs to environmental conditions associated with the delivery system employed. Various means of achieving localized delivery of protein drugs have been investigated and include the use of liposomes, polymer gels, and biodegradable microspheres. Problems with some of these prior delivery systems include inability to maintain protein stability, relatively short drug release durations, inefficient drug loadings, and unsustained and/or incontrollable release rates. The latter may be manifested as a large amount of peptide or protein drug released immediately upon immersion of the delivery device into an aqueous medium. This burst effect can be deleterious to the patient if the drug is potent. Such prior delivery systems may subject proteins to conditions leading to aggregation, denaturation and adsorption at interfaces, deamidation, isomerization, cleavage, oxidation, thiol disulfide exchange, and β elimination in aqueous solutions. The major factors affecting these changes are mechanical forces such as shear, the presence of surfactants, buffers, ionic strength, the presence of oxidizers such as ions, radicals and peroxide, light, pH, temperature, and material surface interactions. Protein denaturation may result in a loss of potency and the conformation changes in the protein molecule may make the protein immunogenic.

For example, polymeric microspheres have been developed that are capable of delivering a virtually constant amount of an encapsulated protein (Takada et al., J. Control. Rel. (1994) 32, 79-85; Sah et al., Journal of Applied Polymer Science (1995) 58, 197-206; Mehta et al., J. Control. Rel. (1996) 41:249-257). This approach has been investigated for local and systemic protein and peptide delivery (Sabel et al., Annals of Surgical Oncology (2004) 11:147-156; Mullerad et al., Cancer Investigation (2003) 21:720-728; Egilmez et al., Cancer Research (2000) 60:3832-3837; Jiang et al., Pharmaceutical Research (2003) 20:452-459). These formulations generally consist of poly(lactide-coglycolide) (PLG), throughout which the protein is distributed as solid particles. The protein is released in three phases: an initial burst; diffusion controlled release; and polymer erosion controlled release. The initial burst is due to surface resident protein particles, while the diffusion controlled release is a result of dissolved protein diffusing through the water-filled pores and channels within the microspheres. To obtain a constant release rate from PLG microspheres, the diffusion phase must overlap with the erosion release phase such that new pores or channels are created during drug release. Polymeric microspheres have one or more of the following advantages of not only providing a constant release, but of being easily injected to the target site, providing a long term release duration, consisting of proven biocompatible materials, having a reasonable shelf-life and degrading to completely bio-resorbable compounds.

However, due to the need for the overlapping polymer erosion phase, a significant problem with polymeric microspheres as a delivery system is maintenance of protein and peptide stability (van de Weert et al., Pharm. Res. (2000) 17:1159-1167). When polymers such as PLG degrade, they liberate acidic oligomers and monomers. The presence of these acids has been found to decrease the local pH at the surface of the polymer and in the pores and channels of the device (Mader et al., Biomaterials (1996) 17:457-461; Fu et al., Pharm. Res. (2000) 17:100-106). In fact, the pH at the centre of a PLG microsphere has been determined to be as low as from 1.5 (Fu et al., Pharm. Res. (2000) 17:100-106) to 1.8 (Shenderova et al., Pharm. Res. (2997) 14:1406-1414). At this pH, many proteins undergo backbone cleavage and deactivation. This reduction in the pH of the inner environment of the microspheres has been linked to inactivation and denaturation of other proteins within PLG microspheres (Park et al., J. Control. Rel. (1995) 33:211-222; Johnson et al., J. Control. Rel. (1991) 17:61-67; Takahata et al., J. Control. Rel. (1998) 50:237-246; Zambaux et al., J. Control. Rel. (1999) 60:179-188; Tabata et al., Pharm. Res. (1993) 10:487-496; Aubert-Pouessel et al., Pharm. Res. (2002) 19:1046-1051). Attempts to overcome this pH issue have included the incorporation of basic salts into the matrix (Zhu et al., Nature Biotechnology (2000) 18:52-57). However, a recent paper, wherein the micro-environmental pH of different size distributions of PLG microspheres was mapped, has demonstrated that the inclusion of a basic excipient does not prevent the internal pH of the microspheres from dropping significantly over a 3 week period (Li et al., J. Control. Rel. (2005) 101:163-173). Moreover, protein-loaded microspheres that have been used in the studies to date have been prepared using techniques such as double emulsification that typically result in protein denaturation (van de Weert et al., Pharm. Res. (2000) 17:1159-1167).

The invention is particularly advantageous for the delivery of peptide and protein drugs, as the above-noted problems associated with environmental conditions are avoided. The protein delivery device of the invention overcomes such problems by providing a polymeric delivery system capable of long-term, relatively constant protein delivery from a biodegradable and biocompatible elastomer device. The elastomer minimizes or avoids acidic degradation of a protein incorporated therein, because the elastomer and its degradation products are not acidic and are biocompatible. That is, the poly(caprolactone) homopolymer used in the elastomer of the invention degrades more slowly and produces fewer acidic degradation products per molecular weight than do other biodegradable polymers, such as poly(lactide-co-glycolide). These properties provide a more suitable pH environment for protein stability within the polymer. Thus, the protein released is more likely to be bioactive and non-immunogenic. Continuous release from the elastomer is achieved by employing an osmotic mechanism and a balance of polymer physical properties with polymer degradation. Aggregation of the protein within the delivery device is minimized or avoided by incorporating the protein as a solid lyophilized with appropriate agents. Use of lyophilization agents provides a driving force for an osmotic drug delivery mechanism. Use of the photo-cross-linked elastomer of the invention allows the device to be fabricated at, e.g., room temperature, thereby avoiding heat which can denature a protein.

Further, the invention substantially reduces or eliminates the burst effect discussed above, due to the rapid setting of the polymer network. The rapid setting is achieved by photo-cross-linking during the manufacturing process, which prevents migration of the peptide or protein drug particles to the polymer surface. Others have attempted to reduce the burst effect by encapsulating the drug in a blend of a hydrophilic polymer with a hydrophobic polymer (Yeh et al., J. Control. Rel. (1995) 37:1-9; Jiang et al., Pharm. Res. (2001) 18:878-885). In that approach, the presence of the hydrophilic polymer reduced the formation of protein crystals at the device surface. However, a combination of reduced burst effect, nearly constant release, low initial drug loading in the device, complete drug entrapment and enhanced total drug released was not demonstrated.

Wu et al. (Journal of Biomatedals Science-Polymer Edition (2003) 14:777-802) used a photo-cross-linkable star polymer combined with poly(ethylene glycol)diacrylate to produce a cross-linked network containing a model protein drug. In that study a star polymer (star-poly(ε-caprolactone)) was used, rather than a star co-polymer as in the invention, resulting in a highly crystalline (38% crystallinity) polymer network, which in turn resulted in very long polymer degradation times (over a period of years) and very slow release of drug. Additionally, a large protein, bovine serum albumin, was incorporated in a co-solvent for both the polymers and the protein. This resulted in a significant protein release burst effect during the initial stage of release (13 to 45% within the first 24 hours). A disadvantage of this approach is potential denaturation of the protein during the free radical cross-linking reaction to prepare the delivery device. Finally, a combination of low initial burst and constant release was not achieved with the formulations of Wu et al. (2003).

The principle of osmotic drug delivery has previously been demonstrated in a delivery system capable of delivering a variety of proteins at the same, almost constant release rate (Amsden et al., J. Control. Rel. (1995) 33:99-105). The proteins were released at the same rate because the driving force for release was the same in each case: the osmotic pressure generated by an inorganic salt. However, use of such salt should preferably be avoided because of its destabilizing effect on a protein and the potential for tissue irritation. The necessary polymer properties for this release mechanism are a radial extension ratio of greater than 1.05, a water permeation coefficient of between 10⁻⁹ and 10⁻¹² g cm/cm² sec cm Hg, a degradation time of greater than 1 month, and minor tissue irritation and inflammation upon implantation. In the previous work, non-degradable polymers such as silicone and poly(ethylene-co-vinyl acetate) were used. With such polymers a device geometry having a constant cross-sectional area is required in order to provide a constant release rate, because the osmotic rupturing mechanism proceeds in a serial manner from the surface to the interior of the device. As one moves from the exterior of the device, usually cylindrical in shape, to the interior, fewer and fewer drug capsules exist within each rupturing layer. This reduction in the number of capsules produces a declining release rate with time.

However, this problem is overcome by the biodegradable elastomers of the invention. Due to their biodegradable nature, their mechanical properties change with time. This property produces a drug-loaded device exhibiting a constant release rate. Although the mass of drug per cross-sectional area of the device is difficult to manipulate, the time required to produce a rupture of the elastomer is more easily manipulated. This latter parameter is determined by the extension ratio and Young's modulus of the polymer. Thus, according to the invention, the elastomer can be tailored such that its Young's modulus decreases with time while the extension ratio remains essentially constant during the release period without significant polymer degradation, such that the time required to rupture the polymer decreases with time. So long as this decrease keeps pace with the decrease in the mass of drug per cross-sectional area of the device, a constant release rate is achieved.

In one embodiment, an osmotic excipient is used in the protein delivery device. The excipient reduces protein aggregation and enhances osmotic protein delivery. Examples of suitable excipients include, but are not limited to, polyols (e.g., trehalose, polyethylene glycol, glycerin, mannitol) and small, neutral amino acids, and combinations thereof. Polyols are preferable because they can generate significant osmotic pressures and are highly effective at preventing protein aggregation. They accomplish this by re-ordering the water around the protein molecule, exerting pressure to reduce the surface contact between the protein and the solvent. This pressure forces hydrophobic portions of the protein to become further removed from the solvent, thus decreasing the likelihood of a hydrophobic-hydrophobic interaction leading to aggregation. Thus, in accordance with the invention, the protein is combined with an excipient by, for example, lyophilization. The ratio of excipient to protein can range from 1:1 to 99:1, depending on the specific conditions. A suspension of the protein/excipient is added to the photo-cross-linkable polymer of the invention prior to cross-linking, and is contained with in the elastomer upon cross-linking.

All cited documents are incorporated herein by reference in their entirety.

The invention is further described in the following non-limiting Examples.

### EXAMPLE 1

### Delivery of Vitamin B12 as a Drug Analog

In this study, we examined an amorphous hydrophobic star co-polymer co-cross-linked with a hydrophilic polymer (poly(ethylene glycol)diacrylate) to yield networks having less than 30% poly(ethylene glycol)diacrylate, and incorporated a low molecular weight drug analog as solid particles during the free radical cross-linking reaction. Vitamin B12 was used as the drug analog because it has a molecular weight (1355 g/mol) similar to that of many peptide drugs, and is readily detectable due to its red color. The loading of vitamin B12 was kept to 1 w/w %, and means of modulating its release from the cylindrical matrix were investigated.

Materials and Methods

D,L-lactide (99%) was obtained from Purac (The Netherlands) and used as received, while ε-caprolactone was obtained from Lancaster (Canada), dried over CaH₂ (Aldrich, Canada) and distilled under vacuum in a nitrogen atmosphere. Other chemicals used were stannous 2-ethylhexanoate, glycerol, acryloyl chloride, triethylamine, 4000 g/mol poly(ethylene glycol)diacrylate (PEGD), 4-dimethylaminopyridine, and 2,2-dimethoxy-2-phenyl-acetophenone, which were all obtained from Aldrich, Canada. Other chemicals used included dichloromethane and ethyl acetate obtained from Fisher, Canada.

Polymer Synthesis

The photo-cross-linkable star-poly(ε-caprolactone-co-D,L-lactide) was prepared as described previously (Aoyagi et al., J. Control. Rel. 1994, 32:87-96; Amsden et al., Biomacromolecules 2004, 5:2479-2486). Briefly, 50:50 molar ratio co-polymers were prepared of molecular weights of 1000, 2700 and 3900 g/mol by melt ring-opening polymerization of ε-caprolactone and D,L-lactide at 140° C. for 24 hours initiated by glycerol and catalyzed by stannous 2-ethylhexanoate. This process yielded a 3-armed star co-polymer terminated in hydroxyl groups. The star co-polymer termini were esterified using acryloyl chloride in anhydrous dichloromethane containing triethylamine as an HCl scavenger and 4-dimethylaminopyridine as a catalyst, at room temperature under nitrogen for 48 hours. Purification yielded an acrylated star co-polymer (ASCP) having a degree of acrylation greater than 85% (Amsden et al., Biomacromolecules 2004, 5:2479-2486).

Device Preparation

Vitamin B12 as received was ground and sieved into less than 100 μm or less than 25 μm fractions. Vitamin B12 loaded cylinders were prepared by first dispersing the vitamin B12 particles in a solution of ASCP dissolved in different amounts of ethyl acetate. In this solution was also dissolved varying amounts of PEGD and 0.015 mg 2,2-dimethoxy-2-phenyl-acetophenone (UV photo-initiator) per gram star co-polymer. The vitamin B12 particles were suspended by agitation using a vortexer, and the suspension quickly poured into sealed glass tubing. The tube was placed into a holder and rotated horizontally at 40 rpm under a long-wave Black-Ray AP UV lamp at an irradiation intensity of 10 mW/cm² for 5 minutes. One end of the tube was then opened to allow for solvent evaporation. Cylinders of length 1 cm were cut from these master cylinders and used in subsequent release experiments.

Polymer Characterization

Thermal properties of the polymers were measured using a Seiko 220U differential scanning calorimeter (DSC) calibrated with indium and gallium standards. 10 mg samples were subjected to a heating-cooling-heating cycle from ambient to 100° C. to −100° C. and back to 100° C. at a rate of 10° C./min. All measurements were taken from the second heating cycle. The molecular weights of the ASCP were measured using a Waters Breeze GPC system connected to a Precision Detectors PD 2000 DLS light scattering detector supplied with a Waters 410 Differential Refractometer. The mobile phase consisted of THF at a flow rate of 2 ml/min with the system at 30° C. The concentration of the polymers used for the GPC measurements were 5 mg/ml and the injection volume was 50 μl. The column configuration consisted of an HP guard column attached to a Phenogel linear (2) 5μ GPC column. The incremental refractive index (dn/dc) was determined using a Wyatt Optilab refractometer at 30° C. and found to be 0.064. Sol contents were measured using dichloromethane extraction at 40° C. on a Soxhlet apparatus. Fourier transform infra-red spectroscopy (FTIR) of the ASCP, the PEGD, cross-linked ASCP, cross-linked PEGD and co-cross-linked ASCP and PEGD was performed by forming a thin film of the polymers directly onto the surface of a KBr crystal. The spectra were collected on a Nicolet XX IR spectrometer.

Release Studies

The vitamin B12 loaded cylinders were placed in 2 ml polypropylene vials containing 1 ml pH 7.4 phosphate buffered saline (per 100 ml:0.16 sodium bisphosphate, 0.758 g sodium phosphate, 0.44 g sodium chloride). The vials were placed on a rotary shaker maintained at 37° C. in an incubator oven. At each sampling period, the 0.5 ml of release medium was removed and replaced with fresh buffer. Vitamin B12 concentration in the release media was measured at 381 nm using a Spectromax microplate spectrophotometer. For every formulation examined the release of 3 cylinders was measured and averaged. The error bars shown in the Figures represent one standard deviation about the mean of this average.

Network Degradation Studies

Vitamin B12-free cylinders were prepared in the same fashion as described above. The initial mass and dimensions of the cylinders were recorded. The cylinders were immersed in 4 ml pH 7.4 phosphate buffered saline maintained at 37° C. in 5 ml glass vials. The buffer was replaced weekly. At given time points, the cylinders were removed, wiped dry with Kim Wipes, their dimensions recorded using calipers, and weighed. Three cylinders were also then dried in a vacuum oven for 48 hours in the presence of dessicant, and weighed dry.

Statistics

Unless otherwise stated, all experiments were performed in triplicate, with the data points in the figures representing the average, and the error bars one standard deviation from the average.

Results

As vitamin B12 absorbs within the UV region, it was important to determine whether the cross-linking conditions affected the vitamin B12. The vitamin B12 was therefore suspended in ethyl acetate in the presence of the photo-initiator, and in a non-acrylated polymer solution also containing the photo-initiator, and subjected to 10 mW/cm² long-wave UV irradiation for 5 minutes. The vitamin B12 was then filtered from solution, dried, and dissolved in varying concentrations and their absorbance measured and compared to that of solutions prepared from the as-received vitamin B12. The results indicated that there was no significant change in the absorbance of the vitamin B12 due to this procedure.

In the following discussion, “ASCP” refers to acrylated star co-polymer, while the number following refers to the molecular weight of the polymer. For example, ASCP 1000 refers to the star co-polymer of molecular weight 1000 g/mol. The thermal characteristics (heat flow as a function of temperature) and thermal properties (glass transition temperature T_{g}, onset of melting point Tₘ, and latent heat of fusion AH) of the networks prepared from these prepolymers and of a network prepared using just PEGD were determined.

The PEGD network did not exhibit a glass transition temperature over the range of temperatures examined; however it did possess a distinct melting endotherm that began at 34° C. The networks prepared without PEGD were amorphous elastomers with glass transition temperatures well below physiologic temperature. The T_{g} decreased as the ASCP prepolymer molecular weight increased, ranging from 4° C. for networks prepared using ASCP 1000 to −8° C. for those prepared using ASCP 3900. As the weight fraction of PEGD incorporated into the networks increased, the T_{g} decreased, and a small melting endotherm appeared. The latent heat of fusion of the melting endotherm increased, and the onset temperature of melting approached that of PEGD as the PEGD content increased. From these data, it can be inferred that at low PEGD concentrations, a homogeneous co-polymer network is formed, wherein the ASCP and the PEGD are co-cross-linked together. As the PEGD concentration in the network increases, regions of solely PEGD are formed within the polymer matrix.

FTIR spectrum analysis showed that the double bonds were completely consumed during the cross-linking reaction. The C═C stretch at 1635 cm⁻¹, which was visible in the uncross-linked ASCP prepolymer and PEGD, disappeared upon exposure to UV irradiation. This was supported by the very low sol contents of the networks formed (values ranged between 2±1% sol for 100% PEGD and 8±2% sol for ASCP 1000 with 20 PEGD).

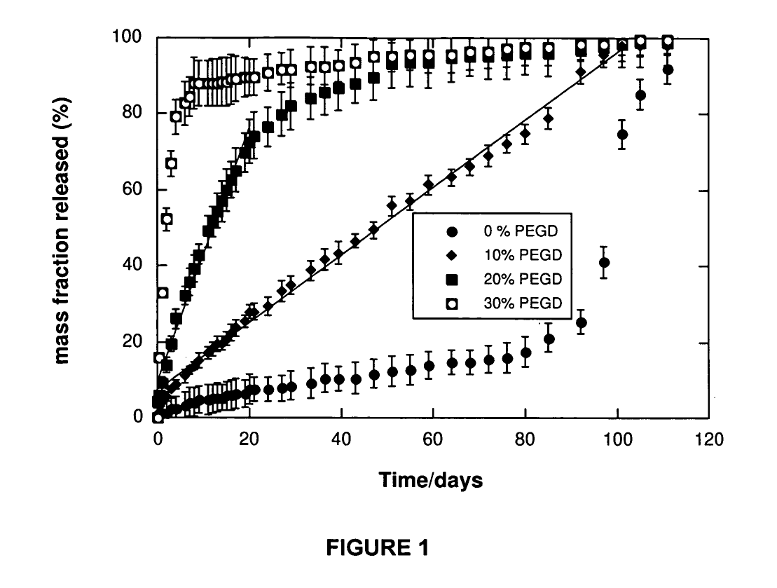

The influence of mass percent PEGD incorporated into the matrix, the diameter of the cylinder, ASCP molecular weight, and particle size of the solid vitamin B12 entrapped within the cylinder on vitamin B12 release were all examined. FIG. 1 illustrates the effect of increasing the mass percent of PEGD in the matrix on vitamin B12 release for matrices prepared using ASCP 1000. The cylinder diameter in this case was 3.5 mm and the vitamin B12 particle size in the cylinders was less than 100 μm. Without any PEGD incorporated into the polymer matrix, vitamin B12 release proceeded very slowly, with less than 20% of the initially loaded amount released over 80 days. By day 96, the release began to accelerate and nearly complete release was obtained by day 111. This release pattern is typical of degradation-controlled release from hydrolytically degradable polymers. As the content of PEGD incorporated into the polymer matrix increased, the release rate of vitamin B12 increased. Cylinders containing 30 w/w % PEGD released approximately 90% of the vitamin B12 within 10 days, while those containing 20 w/w % PEGD reached 90% released by day 45, and those containing 10 w/w % PEGD reached 90% released by 92 days. There was little to no burst effect observed regardless of the weight percent of PEGD in the cylinders. Moreover, for a portion of the release period, the release of vitamin B12 from the cylinders containing 10 w/w % and 20 w/w % PEGD could be approximated as zero order. For example, a linear regression of the data from day 1 to day 100 for the cylinders containing 10 w/w % PEGD resulted in a correlation coefficient (R²) of 0.995, while a linear regression of the data from day 1 to day 20 for cylinders containing 20 w/w % PEGD resulted in a correlation coefficient of 0.981.

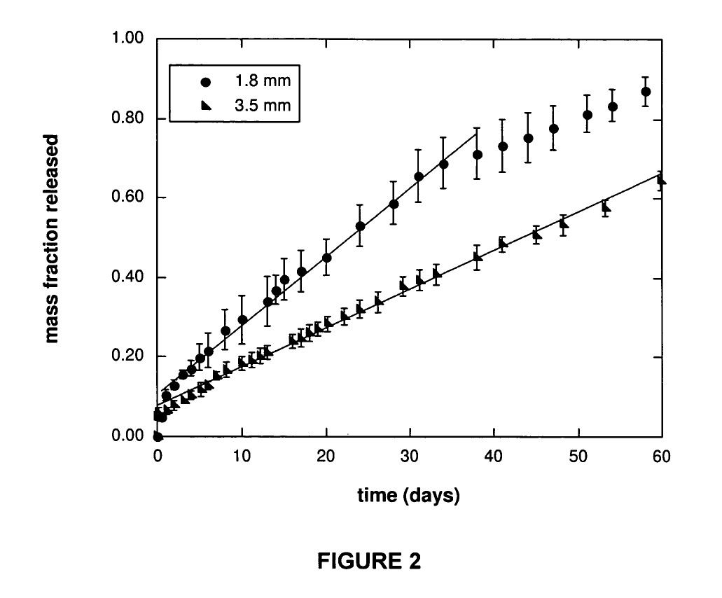

When the cylinder diameter was decreased to 1.8 mm, all other parameters kept constant, the release of vitamin B12 increased (FIG. 2). Again, a period of release was observed that could be approximated as constant, however, the duration of constant release decreased. Linear regression of the data from day 1 to day 35 resulted in a correlation coefficient in this case of 0.993. The rate of release, determined from the slope of the linear portion of the release, was roughly double (0.017 mass fraction released/day) for the 1.8 mm diameter cylinders compared to that for the 3.5 mm diameter cylinders (0.0089 mass fraction released/day).

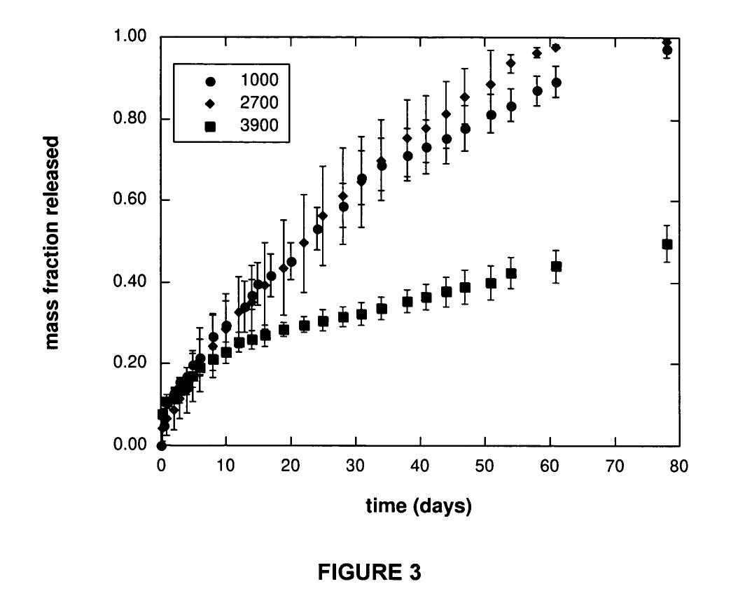

The influence of the molecular weight of the ASCP prepolymer on vitamin B12 release from cylinders containing 10 w/w % PEGD can be seen in FIG. 3. The vitamin B12 particle size was less than 100 μm and the cylinder diameter was 1.8 mm. The release rates are statistically equivalent for the cylinders fabricated with ASCP 1000 and ASCP 2700. For cylinders made with ASCP 3900, the initial release rate is the same as for those made with ASCP 1000 and ASCP 2700 up until day 10, after which release becomes much slower although it continues to be approximately constant.

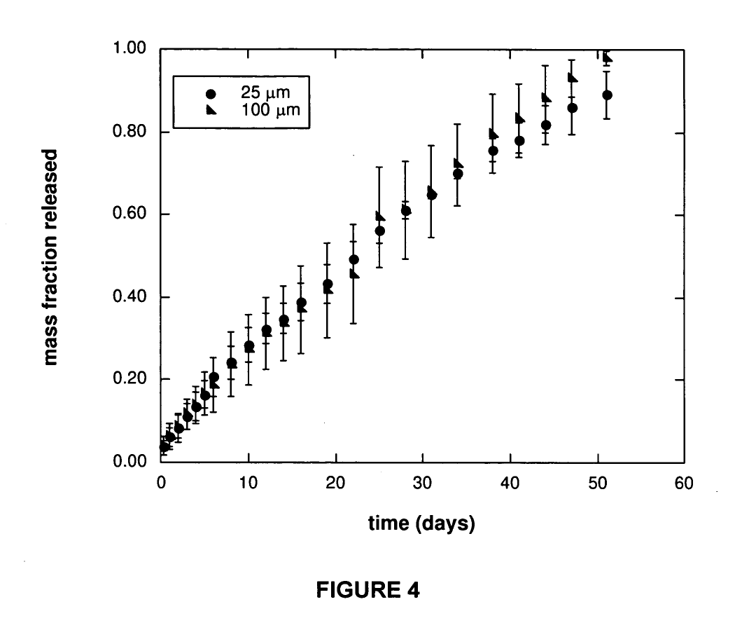

To determine the influence of solid vitamin B12 particle size entrapped within the matrix on its release, cylinders were prepared using ASCP 2700 containing 10 w/w % PEGD. The cylinders had a diameter of 1.8 mm. The results can be seen in FIG. 4. There was no statistical difference in the release pattern of vitamin B12 with respect to its initial particle size in the cylinder.

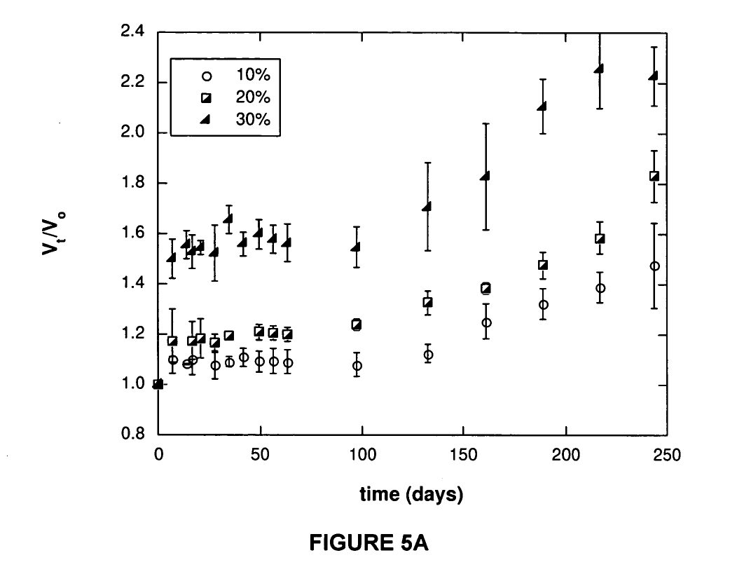

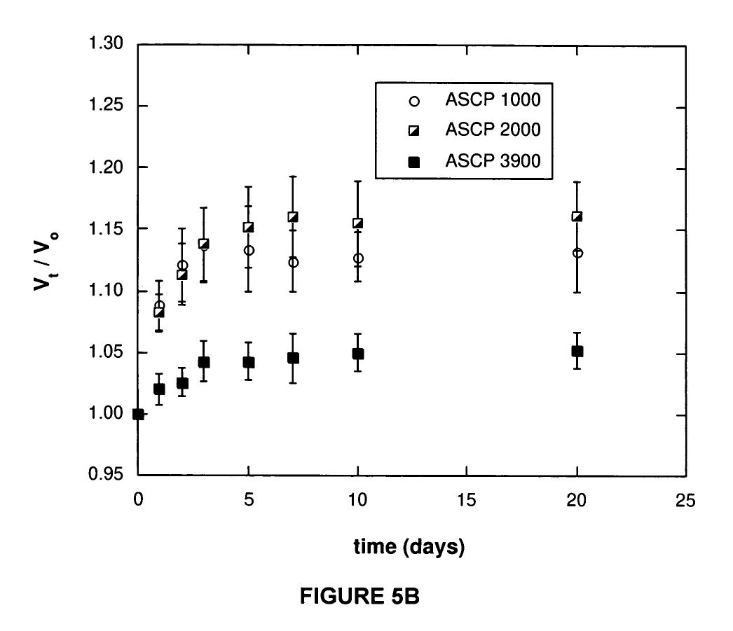

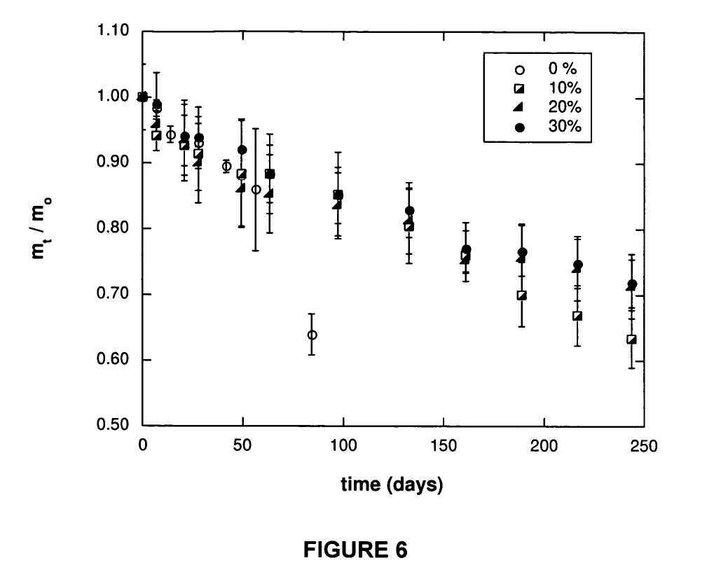

The degradation rate of the networks were determined in vitro and are displayed in terms of the volumetric change and dry mass change with time in FIGS. 5 and 6, respectively. For cylinders prepared with ASCP 1000, the network swelled to an initial maximum within 7 days, and the maximum obtained increased with increasing w/w % PEGD in the network (FIG. 5A). The initial degree of swelling was small, ranging from roughly 7 v/v % for the 10 w/w % PEGD networks to 14 v/v % for the 30 w/w % PEGD networks. The volume of the cylinders remained constant at this initial maximum until day 135. After this time, the cylinders began to swell markedly. The swelling behavior of networks prepared using varying ASCP molecular weight and 10 w/w % PEGD are shown in FIG. 5B. Again, maximal swelling is obtained within 7 days, with the ASCP 1000 and ASCP 2700 reaching essentially the same swelling extent, while the networks containing ASCP 3900 swelled the least.

The mass loss of the ASCP 1000 networks, on the other hand, decreased in a continual, and apparently constant, manner (FIG. 6). The rate of mass loss was the same regardless of the PEGD content of the cylinders, with the exception of the cylinders containing no PEGD. These cylinders lost mass at the same rate as those containing PEGD up until day 49, and then began to degrade more quickly than those containing PEGD. Thus, it would appear that network degradation does not play a dominant role in determining the rate of vitamin B12 release, and that the presence of the PEGD in the matrix modulates the degradation of the elastomer.

Discussion

The work presented indicates that a near-linear release period can be achieved through the co-cross-linking of an amorphous hydrophobic polymer with a hydrophilic polymer to entrap solid drug particles in a cylindrical geometry. The drug loading achieved is low (i.e. less than 5 v/v %). The release rate is independent of the entrapped drug analog particle size, and of the molecular weight of the hydrophobic polymer, at least when it is less than that of the hydrophilic polymer. Furthermore, there is little to no burst effect. The method of manufacture of the delivery system results in 100% drug entrapment efficiency, and can be adapted to geometries other than cylindrical.

The mechanism of release has not been clearly elucidated, but possibilities can be inferred from the data presented. The cylinders swell to an essentially constant volume within the first week, which is maintained during the entire release period. This swelling is driven by the PEGD content of the matrix. There is only a small mass loss during the release period. For example, a mass loss of only approximately 15% occurred over the 100 days of nearly constant release for the cylinders prepared with ASCP 1000 and 10 w/w % PEGD, and a mass loss of only approximately 8% over the 20 days of nearly constant release for the cylinders prepared with ASCP 1000 and 20 w/w % PEGD. Thus, the degradation of the polymer, to generate a greater matrix porosity and thus an increase in solute diffusivity within the matrix, would seem to play only a minor role in the release kinetics. It has been suggested by van Dijkhuizen-Radersma et al. (Biomaterials (2002) 23:1527-1536) who examined vitamin B12 release from poly(ethylene glycol)/poly(butylenes terephthalate) multiblock co-polymers, that a nearly constant release period is a result of a vitamin B12 solubility limitation within the swollen matrix. However, if dissolution of vitamin B12 within the matrix was rate-limiting, then decreasing the particle size of the vitamin B12 should have had an influence on the release rate, which was not observed in this work. Another possibility is that the release is driven by the osmotic pressure generated by the polymer enveloped vitamin B12 particles within the matrix. This release mechanism has been shown to be capable of generating constant release from cylindrical devices (Amsden et al. J. Control. Rel. (2003) 93:249-258; Gu et al., J. Control. Rel. (2005) 102:607-617; Schirrer et al., J. Mater. Sci. (1992) 27:3424-3434). In this situation, water is drawn into the polymer matrix due to the osmotic activity of the solute, and the pressure generated creates microcracks within the matrix through which the dissolved solute is forced out. In the present situation, the PEGD incorporated may act to enhance the rate of water uptake while at the same time providing aqueous pathways for the movement of the solute to the surface. At present, the release mechanism is not clear, and may be due to a combination of all the mechanisms discussed.

### EXAMPLE 2

### Demonstration of Effect of PEG Molecular Weight on Release Rate

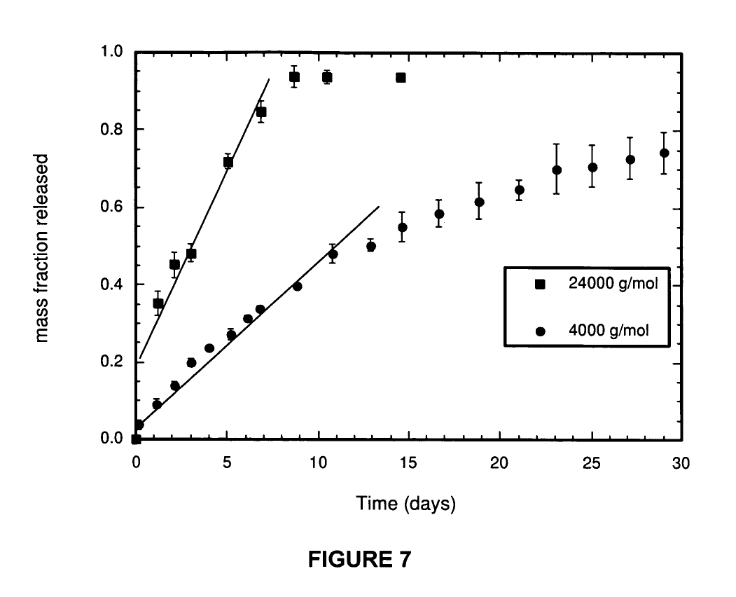

Acrylated star-poly(ε-caprolactone-co-D,L-lactide) (ASCP) of molecular weight 2700 g/mol was co-dissolved in tetrahydrofuran with poly(ethylene glycol)diacrylate (PEGD) of molecular weight 4000 g/mol or 24000 g/mol. The total polymer concentration was 1 g/mL THF. The mass ratio of PEGD:ASCP was 1:9 (10% PEGD). In this solution was suspended vitamin B12 particles that had been ground and sieved to less than 100 μm in diameter. The total mass of vitamin B12 to polymer was 1:99 (1% vitamin B12). 1.5 w/w % of 2,2-dimethoxy-2-phenyl-acetophenone (DMPA) photoinitiator was also included. The suspension was injected into a 1.8 mm diameter glass tube to a length of 34 cm and sealed with parafilm. The tube was then held and rotated slowly by hand under a long-wave Black-Ray AP UV lamp with a filter of 220 nm to 480 nm at an irradiation intensity of 100 W/cm² for 5 minutes. The parafilm was removed to allow for solvent evaporation. Cylinders of 1 cm length were cut from these master cylinders and used in subsequent release studies. In vitro release studies were performed by placing the cylinders in polypropylene vials containing 1 mL of pH 7.4 PBS. Three cylinders were used for each formulation. The vials were placed on a rotary shaker inside an incubator maintained at 37° C. At each sampling period, 0.5 mL samples were taken and replaced with fresh PBS. The concentration of vitamin B12 in the release samples was measured with a Spectromax microplate spectrophotometer. 0.250 mL of vitamin B12 sample per well was added into a 96 well microplate, and the absorbance of these samples was read at 381 nm. The release results are shown in FIG. 7. As the PEGD molecular weight increased, the release rate increased, yet remained approximately constant for a substantive time period.

### EXAMPLE 3

### Influence of Drug Solubility on Release Rate

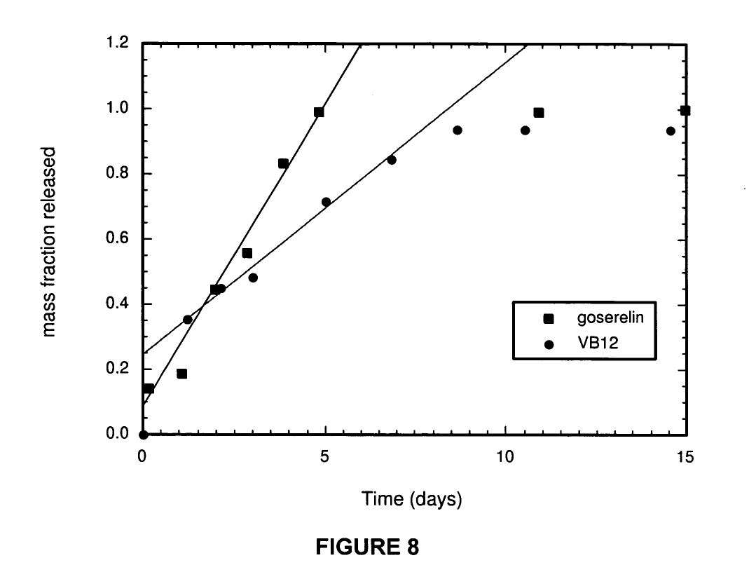

Goserelin acetate and vitamin B12 were incorporated into cylindrical polymer matrices as described in Example 2, but using only PEGD 24000 g/mol. Goserelin acetate is a peptide having the sequence Pyr-His-Trp-Ser-Tyr-D-Ser(tBu)-Leu-Arg-Pro-Azagly-NH2 acetate salt. Its molecular weight is 1269 g/mol and it has a water solubility of 20 mg/mL at 25° C. The molecular weight of vitamin B12 is 1329 g/mol and it has a water solubility of 12.5 mg/mL. The diffusivities of these two compounds was determined at 37° C. using Pulsed Field Gradient Nuclear Magnetic Resonance. The measured diffusivities were 2.8±0.1 and 2.6±0.1 (10⁻⁶) cm²/s for vitamin B12 and goserelin, respectively. These diffusivities are essentially the same. In vitro release experiments on these cylinders were performed as described in Example 2. The results are given in FIG. 8. The goserelin was released at an appreciably faster rate as indicated by the regression line drawn through the data. As the diffusivities of the two compounds are very similar, and degradation of the polymer occurs slowly, release cannot be controlled by either diffusion or polymer degradation. The higher release rate must therefore be a result of the higher aqueous solubility of the goserelin acetate. Thus, release is driven by the dissolution rate of the compound in the aqueous regions of the polymer matrix.

### EXAMPLE 4

### Elastomer Preparation with Acrylated poly(D,L-lactide)-block-poly(ethylene glycol)-block-poly(D,L-lactide) and ASCP 2700

Polyethylene glycol dihydroxy was used in initiate ring-opening polymerization of D,L-lactide in the presence of an organo-metallic catalyst. An example of the procedure used to prepare the DLPEGDL is as follows: 12.55 g of PEG were added to a flame-dried ampule. The PEG was dried for 12 hours at 100° C. under vacuum to remove traces of water. The PEG was cooled to room temperature under vacuum and 4.25 g of D,L-lactide were added to the ampule. The ampule was then placed in the oven at 140° C. until the PEG and the D,L-lactide had both melted. The ampule was removed from the oven, 0.003 g tin (II) ethylhexanoate was added to the melt and the mixture was vortexed under vacuum. The ampule was then flame-seated and placed in the oven for 24 hours at 140° C. When the polymerization time had elapsed, the polymer was cooled cooled to room temperature, and purified by precipitation. The purification procedure is as follows: 10 g of DLPEGDL was dissolved in 50 ml of distilled dichloromethane. The solution was then precipitated in excess diethyl ether that was cooled in a bath of methanol and dry ice. The precipitate was then filtered and placed under vacuum at room temperature for 3 days to remove solvents. The DLPEGDL was stored under vacuum until further use.

Termini acrylation of the DLPEGDL was performed by esterification using acryloyl chloride. Before acrylation the DLPEGDL was dried under vacuum at 100° C. for 12 hours to remove trace amounts of water or solvents. Following this, the acrylation reaction was carried in distilled dichloromethane with an HCl scavenger triethylamine, and the catalyst 4-dimethyl aminopyridine. A 1:1 molar equivalent of acryloyl chloride to triethylamine was used. The final solution was dried under vacuum and redissolved in ethyl acetate. The precipated HCl-triethylamine salt was removed by filtration. The ethyl acetate was dried from the filtrate and the resulting polymer was resolubilized dichloromethane. The solution was then precipitated in excess diethyl ether that was cooled in a bath of methanol and dry ice. The precipitate was then filtered and placed under vacuum at room temperature for 3 days to remove solvents. The acrylated DLPEGDL (A-DLPEGDL) was stored under at −20° C. until further use.

To prepare elastomer matrices, ASCP 2700, varying weight percentages of A-DLPEGDL and 1.5 weight percentage of photoinitiator 2,2-dimethoxy-2-phenyl-acetophenone were solubilized in dichloromethane (1/1:w/v). The mixture was then drawn into hollow glass cylinders. The cylinders were then exposed to long-wave ultraviolet light at 100 mW/cm² for 10 minutes. The resulting polymer rods were dried under vacuum for 24 hours, removed from their glass cylinders, cut to a length of 1.5 cm and characterized. ¹H NMR analysis of PEG based components was conducted using a Bruker Avance-400 400 MHz autosampling spectrometer. All PEG based samples were prepared d₆-DMSO. Thermal analysis was conducted using a Seiko Instruments DSC200U. Samples were run using a heating, cooling, heating cycle as follows: ambient temperature to 120° C., held 10 minutes, to −100° C., held 10 minutes, to 120° C., held 10 minutes. The rate of heating/cooling was 10° C./min. Sol content tests were performed as follows: initial massing, 2 sequential solubilizations of the rods in dichloromethane for 24 hours each, 24 hours drying under vacuum, and re-massing. Data reported is an average of three samples. The resulting properties of the elastomers are given in Table 1.

TABLE 1
Glass transition temperature and sol content of elastomers
made through co-crosslinking ASCP 2700 and A-DLPEGDL.

| A-DLPEGDL | Glass transition | Sol content |
|---|---|---|
| incorporated (w/w %) | temperature (° C.) | (w/w %) (mean ± S.D.) |
| 10 | −7 | 9.0 ± 2.0 |
| 20 | −11 | 9.2 ± 1.5 |

### EQUIVALENTS

Those skilled in the art will recognize variants of the embodiments described herein and presented in the above Examples. Such variants are intended to be within the scope of the invention and are covered by the appended claims.

1. A degradable elastomer, comprising:
a biocompatible degradable cross-linked network of:
(i) a hydrophobic, hydrolysable amorphous star polymer; and
(ii) a hydrophilic polymer;
wherein one of the hydrophobic polymer or the hydrophilic polymer includes two or more cross-linkable groups on the polymer chain terminus, and the other of the hydrophobic polymer or the hydrophilic polymer includes one or more cross-linkable groups on the polymer chain terminus.

2. The elastomer of claim 1, wherein the star polymer comprises at least one monomer, said at least one monomer capable of forming a degradable linkage to another monomer.

3. The elastomer of claim 2, wherein the at least one monomer is selected from the group consisting of lactones, carbonates, and cyclic amides, and combinations thereof.

4. The elastomer of claim 2, wherein the at least one monomer is selected from valerolactone, caprolactone, dioxepanone, lactide, glycolide, trimethylene carbonate, and O-benzyl-L-serine.

5. The elastomer of claim 1, wherein the star polymer has a glass transition temperature (T_{g}) below room temperature.

6. The elastomer of claim 1, wherein the star polymer comprises star-poly(ε-caprolactone-co-D,L-lactide).

7. The elastomer of claim 1, wherein the hydrophilic polymer is selected from poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates such as hyalyuronic acid, chitosan, dextran, heparan sulfate, heparin, alginate, and proteins such as gelatin, collagen, albumin, ovalbumin, and polyamino acids.

8. The elastomer of claim 1, wherein the hydrophilic polymer comprises poly(ethylene glycol)diacrylate.

9. The elastomer of claim 1, wherein the hydrophobic polymer forms greater than 70% by weight of the total polymer mass.

10. The elastomer of claim 1, wherein the elastomer is biodegradable.

11. A method of preparing a biocompatible degradable elastomer, comprising:
providing a hydrophobic, hydrolysable amorphous star polymer and a hydrophilic polymer, one of the hydrophobic polymer or the hydrophilic polymer including two or more cross-linkable groups on the polymer chain terminus, and the other of the hydrophobic polymer or the hydrophilic polymer including one or more cross-linkable groups on the polymer chain terminus;
combining the hydrophobic, hydrolysable amorphous star polymer and the hydrophilic, biocompatible polymer; and
cross-linking the hydrophobic, hydrolysable amorphous star polymer and the hydrophilic, biocompatible polymer to create a degradable cross-linked elastomer.

12. The method of claim 11, further comprising combining the hydrophobic, hydrolysable amorphous star polymer and the hydrophilic polymer in a mold prior to cross-linking.

13. The method of claim 11, wherein the star polymer comprises at least one monomer, said at least one monomer capable of forming a biodegradable linkage to another monomer.

14. The method of claim 13, wherein the monomer is capable of undergoing polymerization through a ring-opening reaction or a condensation reaction.

15. The method of claim 13, wherein the at least one monomer is selected from the group consisting of lactones, carbonates, and cyclic amides.

16. The method of claim 13, wherein the at least one monomer is selected from valerolactone, caprolactone, dioxepanone, lactide, glycolide, trimethylene carbonate, and O-benzyl-L-serine.

17. The method of claim 11, further comprising forming the cross-linked network through action of an initiator, wherein the initiator absorbs energy to form a free radical which reacts with an allyl group of the cross-linkable group.

18. The method of claim 17, wherein the cross-linkable group comprises a photo-cross-linkable group selected from acrylate, coumarin, thymine, cinnamate, diacrylate, oligoacrylate, methacrylate, dimethacrylate, and oligomethacrylate.

19. The method of claim 18, wherein the initiator is a photo-initiator selected from acetophenone derivatives, camphorquinone, Irgacure® (1-hydroxy-cyclohexyl-phenyl-ketone, 1-[4-(2-hydroxyethoxy)-phenyl]-2-hydroxy-2-methyl-1-propane-1-one, 2,2-dimethoxy-1,2-diphenylethan-1-one, or 2-methyl-1-[4-(methylthio)phenyl]-2-(4-morpho-linyl)-1-propanone, 2,2-dimethyl-2-phenylacetaphenone, 2-methoxy-2-phenylacetaphenone), Darocur® (1-[4-(2-hydroxyethoxy)-phenyl]-2-hydroxy-2-methyl-1-propane-1-one or 2,4,6-trimethylbenzoyl-diphenyl-phosphineoxide), and eosin dye.

20. The method of claim 17, wherein the initiator is a thermal initiator selected from potassium persulfate, with or without tetraamethyl ethylenediamine; benzoylperoxide, with or without triethanolamine; and ammonium persulfate with sodium bisulfite.

21. The method of claim 11, wherein the star polymer comprises star-poly(ε-caprolactone-co-D,L-lactide).

22. The method of claim 11, wherein the hydrophilic polymer is selected from poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates such as hyalyuronic acid, chitosan, dextran, heparan sulfate, heparin, alginate, and proteins such as gelatin, collagen, albumin, ovalbumin, and polyamino acids.

23. The method of claim 11, wherein the hydrophilic polymer comprises poly(ethylene glycol)diacrylate.

24. An implantable delivery system for delivering a pharmaceutical agent to a subject, comprising the degradable elastomer of claim 1 and the agent distributed within the network,
wherein the network provides controlled release of the agent.

25. The implantable delivery system of claim 24, wherein the agent is a therapeutic compound, pharmaceutical, biopharmaceutical, medicament, hormone, peptide, protein, nucleic acid, vector, virus, antigen, or antibody, or combination thereof.

26. The implantable delivery system of claim 24, wherein rate of release of the agent increases as the content of hydrophobic polymer in the network decreases.

27. A device comprising the degradable elastomer of claim 1.

28. The device of claim 27, wherein the device is a biomedical device selected from a needle, stent, catheter, and a scaffold.

29. A method of delivering a pharmaceutical agent to a subject, comprising:
providing the agent in the implantable delivery system of claim 24; and
implanting the delivery system in the subject.

30. The method of claim 29, wherein the agent is a therapeutic compound, pharmaceutical, biopharmaceutical, medicament, hormone, peptide, protein, nucleic acid, vector, virus, antigen, or antibody, or combination thereof.
